(19) United States
(12) Patent Application Publication
(10) Pub. No.: US 2026/0031201 A1
(43) Pub. Date: Jan. 29, 2026

(54) SURVIVAL CURVE GENERATING SYSTEM USING EXPONENTIAL FUNCTION AND METHOD THEREOF

(71) Applicant: AJOU UNIVERSITY INDUSTRY-ACADEMIC COOPERATION FOUNDATION, Suwon-si, Gyeonggi-do (KR)

(72) Inventor: O Yeon CHO, Suwon-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 18/996,993

(22) PCT Filed: Jul. 18, 2023
(86) PCT No.: PCT/KR2023/010259

§ 371 Date: Jan. 18, 2025

(30) Foreign Application Priority Data
Jul. 18, 2022 (KR) 10-2022-0088023
Jul. 11, 2023 (KR) 10-2023-0089918

(51) Int. Cl. G16H 10/20; G06F 17/14

(52) U.S. Cl. CPC G16H 10/20; G06F 17/14

(57) ABSTRACT

The present invention relates to a survival curve generating system using an exponential function and a method thereof. According to the present invention, a survival curve generating system includes a data collection unit that collects data obtained from a clinical trial for cancer, a survival curve construction unit that generates a survival curve by using a survival curve model including multiple exponential functions based on the collected data, and an analysis unit that calculates a relative ratio of one exponential function included in the survival curve model, obtains intensity from a sigmoid curve derived using the relative ratio, and separates a patient group based on the intensity.

### TECHNICAL FIELD

The present invention relates to a survival curve generating system using an exponential function and a method thereof, and more specifically, to a survival curve generating system that may generate a survival curve by using a survival curve model including multiple exponential functions and allow patient groups to be separated from each other through the survival curve and a method thereof.

### BACKGROUND ART

Comparison of survival curves assumes that the risk is constant over time. However, when comparing treatments or biomarkers in reality, a risk difference is not the same at all times. In the case of people who died early or survived for a long time, the relative risk may be high or low due to non-treatment factors. It may be assumed that events occur quickly due to internal factors or do not occur even after a long time, and an example of cervical cancer shows this.

FIG. 1 is a graph illustrating survival curves of a clinical trial comparing disease-free survival rates between chemoradiation and radiation therapy, and FIG. 2 is a graph illustrating assumptions on an event occurrence rate to the expected total event occurrence in a clinical trial for comparing survival rates between chemoradiation and radiation therapy.

As illustrated in FIG. 1, after radiation therapy for cervical cancer, half of the patients die within one year or around two years, and a death rate of the patient with this early progression is 1.8 to 3 times higher than the death rate of a patient who does not receive radiation therapy.

This means that, in clinical practice, there are some patients who progress rapidly after treatment even having the same disease.

Therefore, if the survival curve graph may be modeled based on the collected clinical data and calculate the predicted total event progression rate based thereon as illustrated in FIG. 2, two survival curves may be compared based on the estimated progression of event rather than on time.

However, in clinical trials, restricted mean survival time (RMST) or restricted mean time lost (RMLT) is attempted to overcome violation of the proportional hazard model, which will reflect even the part that violates the proportional hazard assumption, and thus, there are still limitations in evaluating the effectiveness of treatment or biomarkers.

In addition, clinical studies related to cancer treatment to date have a problem of not separately considering early progression and death when observing cancer progression and death.

Technology that serves as the background of the present invention is disclosed in Korean Patent Publication No. 10-2022-0056527 (published on May 6, 2022).

### DISCLOSURE

### Technical Problem

A technical object to be achieved by the present invention is to provide a survival curve generating system that generates a survival curve by using a survival curve model including multiple exponential functions and allows patient groups to be separated from each other through the survival curve and a method thereof.

### Technical Solution

According to the present invention, a survival curve generating system using an exponential function includes a data collection unit that collects data obtained from a clinical trial for cancer, a survival curve construction unit that generates a survival curve by using a survival curve model including multiple exponential functions based on the collected data, and an analysis unit that calculates a relative ratio of one exponential function included in the survival curve model, obtains intensity from a sigmoid curve derived using the relative ratio, and separates a patient group based on the intensity.

In addition, according to the present invention, a survival curve generating method using a survival curve generating system includes a step of collecting data obtained from a clinical trial for cancer, a step of modeling a survival curve by using the collected data, a step of calculating intensity by applying a sigmoid equation to a relative ratio of the survival curve, a step of classifying restricted mean time lost (RMLT) according to a time change, and a step of selecting a section of the survival curve by using time, intensity, and the restricted mean time lost (RMLT).

In the step of modeling the survival curve, the survival curve may be modelled by using a KWW function generated by using an equation below,

$$S ( t ) = α 1 \times exp ( - {t}^{α} ) + β 1 \times exp ( - {t}^{β} ) + e ( β \geq 1 ⋂ α > 0 ⋂ β - α > 0 , c = β - α )$$

In the step of modeling the survival curve, a range of a may be set from 0.01 to 10 at 0.01 intervals and a range of β may be set from 1 to 10 at 0.1 intervals.

In the step of calculating the intensity, KWW (A) may be obtained by normalizing α1/exp (−t^{α}), KWW (B) may be obtained by by normalizing β1×exp (−t^{β})+e and then a graph for time may be obtained by inserting the obtained KWW (A) and KWW (B) into

$$\frac{KWW ( B )}{KWW ( A ) + KWW ( B )} .$$

In the step of calculating the intensity, the intensity may be calculated by applying a sigmoid equation of an equation below to a relative ratio for KWW (B),

$$y = d + \frac{a - d}{{( 1 + exp ( b ( x - c ) ) )}^{g}}$$

where a is a maximum value, b is a slope factor, c is a position parameter, d is a minimum value, and g represents an asymmetry factor.

In the step of classifying the restricted mean time lost (RMLT), a specific point in time for an expected value of occurrence for death or relapse may be set, and the restricted mean time lost (RMLT) may be classified into first restricted mean time lost (RMLT1) and second restricted mean time lost (RMLT2) based on the set specific point in time.

In the step of selecting the section of the survival curve, the intensity and the restricted mean time lost (RMLT) may be analyzed to select a section in which there is no difference in the restricted mean time lost (RMLT) for intensities of a comparison group and a contrast group of the survival curve.

### Advantageous Effects

In this way, according to the present invention, the existence of other groups in the existing survival curve may be theoretically suggested, and it is possible to specify groups that are not related to the applied effect in the survival curve of a randomized clinical trial for evaluating a treatment effect of cancer, and thus, an effect of the randomized clinical trial for evaluating the treatment effect of cancer may be accurately and efficiently evaluated.

In addition, according to the present invention, meaningful information on the comparison between a comparison group and a contrast group may be provided by separating a treatment refractory group in results of survival curves, and the groups that do not show statistically significant differences during an observation period may be re-evaluated.

### DESCRIPTION OF DRAWINGS

FIG. 1 is a graph illustrating survival curves of a clinical trial comparing disease-free survival rates between chemoradiation and radiation therapy.

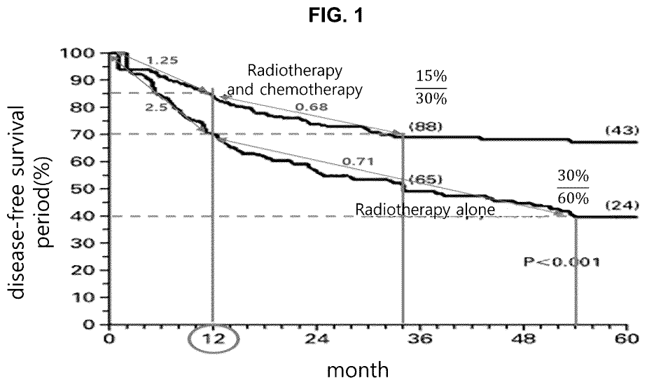

FIG. 2 is a graph illustrating assumptions on an event occurrence rate to the expected total event occurrence in a clinical trial for comparing survival rates between chemoradiation and radiation therapy.

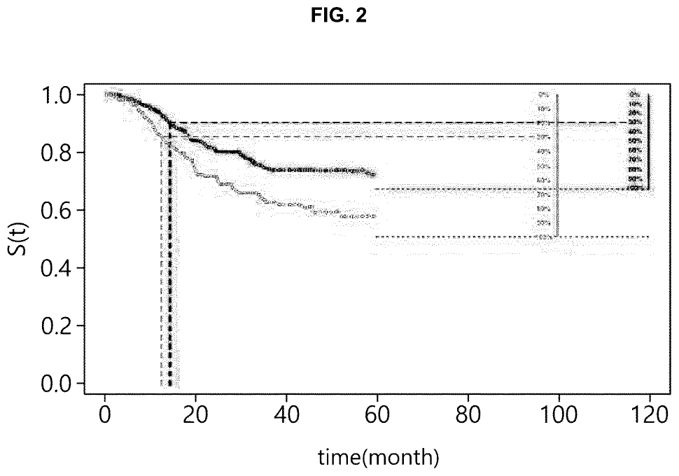

FIG. 3 is a configuration diagram illustrating a survival curve generating system according to an embodiment of the present invention.

FIG. 4 is a flowchart illustrating a survival curve generating method by using a survival curve generating system, according to an embodiment of the present invention.

FIG. 5 illustrates graphs schematically illustrating survival curve prediction models.

FIG. 6 illustrates graphs in the range of α (A) and the range of β (B) in a modified KWW function in step S420 illustrated in FIG. 4.

FIG. 7 illustrates two survival curves when a modified equation is applied in step S430 illustrated in FIG. 4.

FIG. 8 illustrates relative ratios of the graphs illustrated in FIG. 7.

FIG. 9 illustrates graphs of percentages according to a relative ratio of KWW (B) obtained from FIG. 8.

FIG. 10 illustrates examples of a method of acquiring intensity for a period of −3 to 3 from a sigmoid curve derived by using the data collected in a period of 0 to 1.

FIG. 11 illustrates graphs of restricted mean time lost classified in step S430 illustrated in FIG. 4.

FIG. 12 is a graph showing a difference between intensity of a contrast group and a comparison group, the first restricted mean time lost (RMLT1), the second restricted mean time lost (RMLT2), and the restricted mean time lost (RMLT) in a clinical trial for comparing survival rates of chemoradiation and radiation therapy.

FIG. 13 is a graph showing the time according to intensity in the restricted mean time lost illustrated in FIG. 12.

FIG. 14 illustrates graphs obtained by calculating all RMTL and modified RMTL of a contrast group and a comparison group illustrated in FIG. 13.

### BEST MODE FOR INVENTION

Hereinafter, preferred embodiments of the present invention will be described in detail with reference to the attached drawings. In this process, thicknesses of lines and sizes of components illustrated in the drawings may be exaggerated for the sake of clarity and convenience of description.

In addition, the terms described below are terms defined in consideration of their functions in the present invention, and may change depending on the intention or custom of a user or operator. Therefore, definitions of the terms should be made based on the contents throughout the present specification.

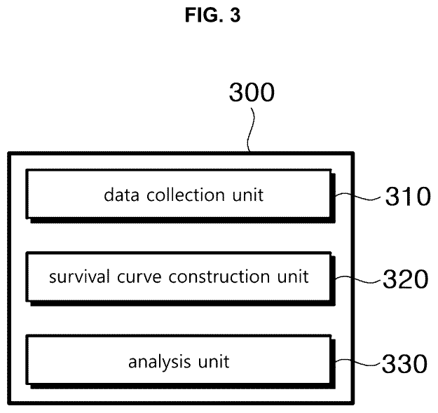

Hereinafter, a survival curve generating system using an exponential function, according to an embodiment of the present invention, will be specifically described with reference to FIG. 3.

FIG. 3 is a configuration diagram illustrating a survival curve generating system according to an embodiment of the present invention.

As illustrated in FIG. 3, a survival curve generating system 300 according to an embodiment includes a data collection unit 310, a survival curve construction unit 320, and an analysis unit 330.

First, the data collection unit 310 collects data obtained from a clinical trial for cancer.

The survival curve construction unit 320 constructs a survival curve model including multiple exponential functions based on the collected data.

Finally, the analysis unit 330 calculates a relative ratio of one exponential function included in the survival curve model, and acquires intensity from a sigmoid curve derived by using the calculated relative ratio. In addition, the analysis unit 330 separates patient groups based on the intensity.

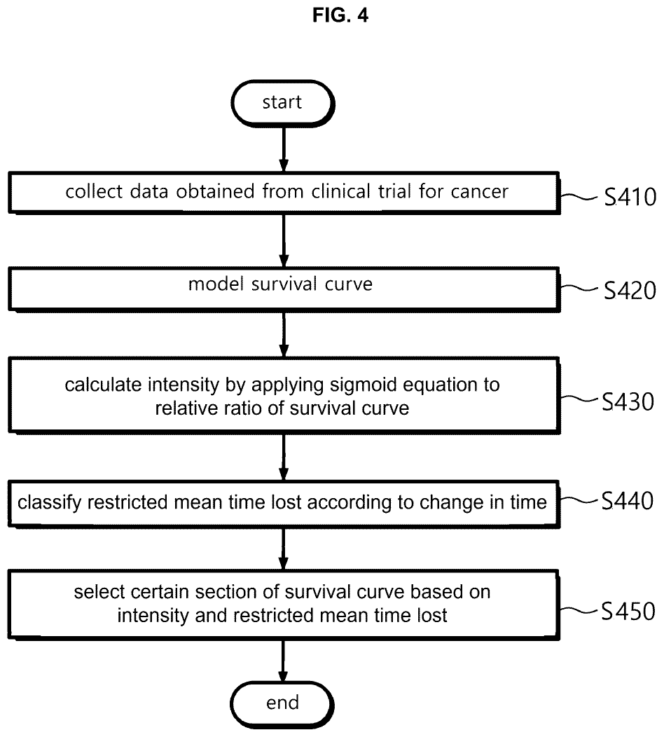

Hereinafter, a method of generating a survival curve by using the survival curve generating system 300, according to an embodiment of the present invention, will be described in more detail with reference to FIG. 4 to FIG. 13.

FIG. 4 is a flowchart illustrating a method of generating a survival curve by using a survival curve generating system according to an embodiment of the present invention.

As illustrated in FIG. 4, the data collection unit 110 collects data obtained from a clinical trial for cancer (S410).

The data obtained here includes at least one of the type of cancer, a patient's survival period, an observation stop state, a patient's age, presence or absence of concomitant diseases, and a treatment application method.

Then, the survival curve construction unit 320 models a survival curve (S420).

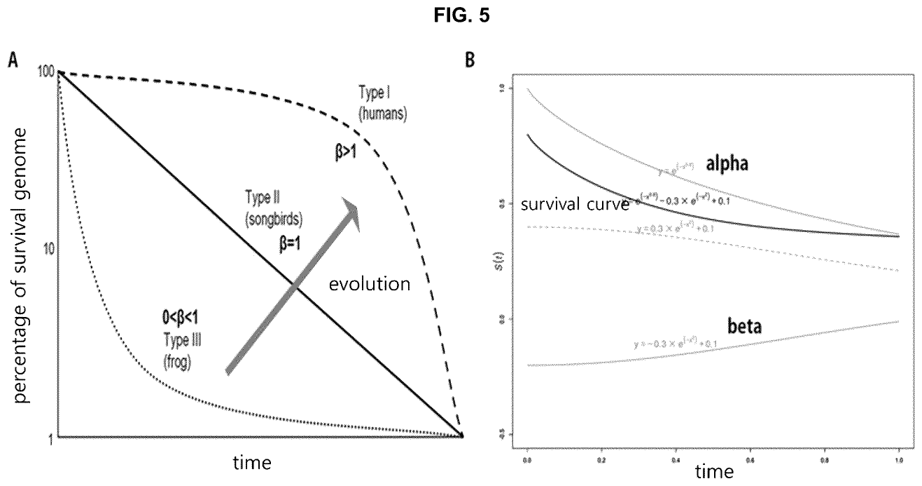

FIG. 5 illustrates graphs, each schematically showing a survival curve prediction model.

As illustrated in FIG. 5 (A), the Kohlrausch-Williams-Watts (KWW) function (S(t)=exp(t^{β})(β>0) well schematically illustrates a survival curve according to evolution.

In addition, as illustrated in FIG. 5 (B), when some patients have decreased body defense ability for some reason, it is assumed that a KWW function β of that group will have a function of less α than that of other groups, and accordingly, it may be assumed as in Equation 1 below.

$$S ( t ) = exp ( - {t}^{α} ) + exp ( - {t}^{β} ) ( α < β ) \qquad [ Equation 1 ]$$

That is, curves of patients who die early or relapse will have a high proportion of exp (−t^{α}) (α>0) and a low proportion of exp(−t^{β}), and a proportion of relative exp(−t^{β}) (β≥1) will increase over time.

In addition, when a relative proportion of exp(t^{β}) is low, the influence of a curve of exp(−t^{α}) increases, and as the relative proportion of exp(−t^{β}) increases, the influence of a curve of exp(−t^{β}) decreases in contrast to a group where incidents occur early, and the group in which additional events do not occur will be quantified.

In other words, the degree of event progression may be quantified through the proportion of exp(−t^{β}) among exp(−t^{α}) and exp(−t^{β}). At this time, weights of the two equations are assumed to be equal to each other.

However, the objective of the present invention is to develop a survival curve model that may specify groups with high or low risk.

Therefore, in the present invention, the KWW function of Equation 1 is transformed as shown in Equation 2 below.

$$S ( t ) = α 1 \times exp ( - {t}^{α} ) + β 1 \times exp ( - {t}^{β} ) + e ( β \geq 1 ⋂ α > 0 ⋂ β - α > 0 , c = β - α ) \qquad [ Equation 2 ]$$

Here, a range of α is set from 0.01 to 10 at 0.01 intervals, and a range of β is set from 1 to 10 at 0.1 intervals.

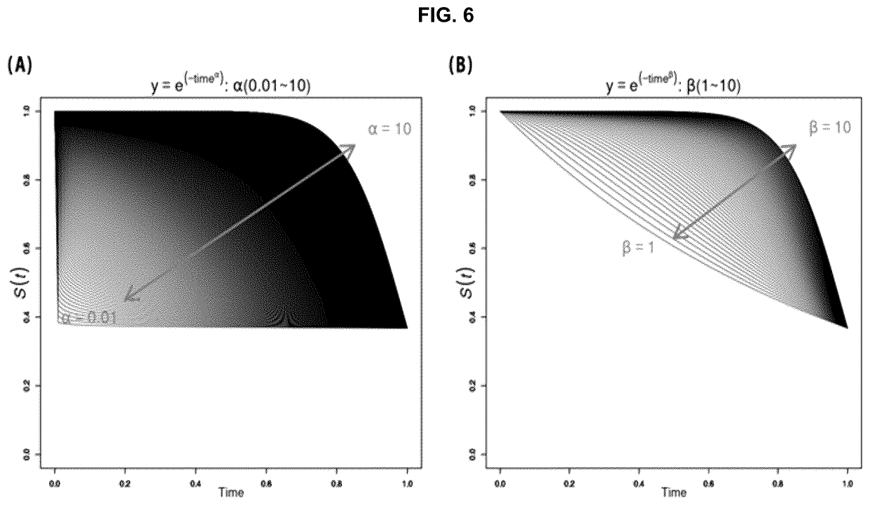

FIG. 6 illustrates graphs in the range of α (A) and the range of β (B) in a modified KWW function in step S420 illustrated in FIG. 4.

As illustrated in FIG. 6, the present invention calculates a determination coefficient R\hat{ }2 in the set ranges of α and β. Then, the present invention extracts α and β corresponding to the smallest value among multiple c values and corresponding to the largest value of determination coefficient R\hat{ }2.

Then, the analysis unit 330 calculates intensity by applying a sigmoid equation to a relative ratio of the survival curve obtained through the modified equation (S430).

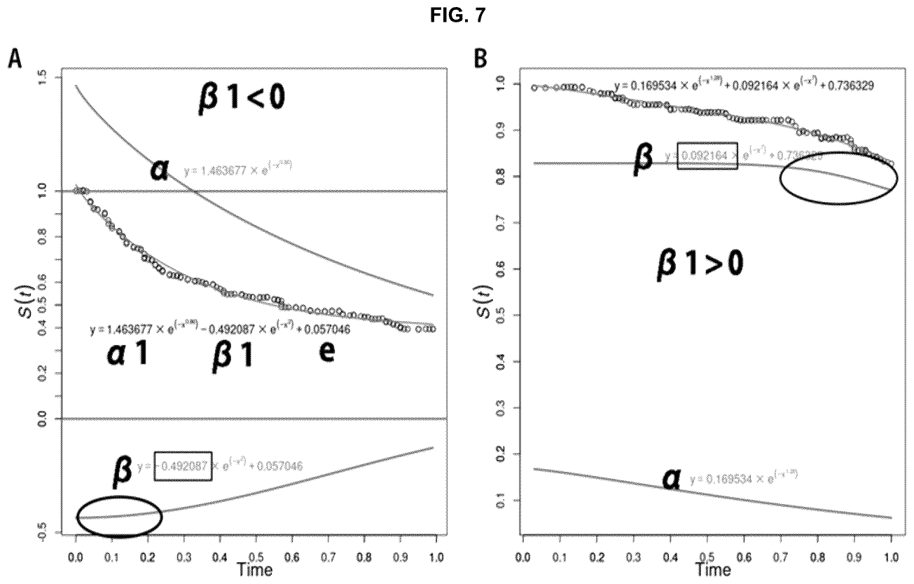

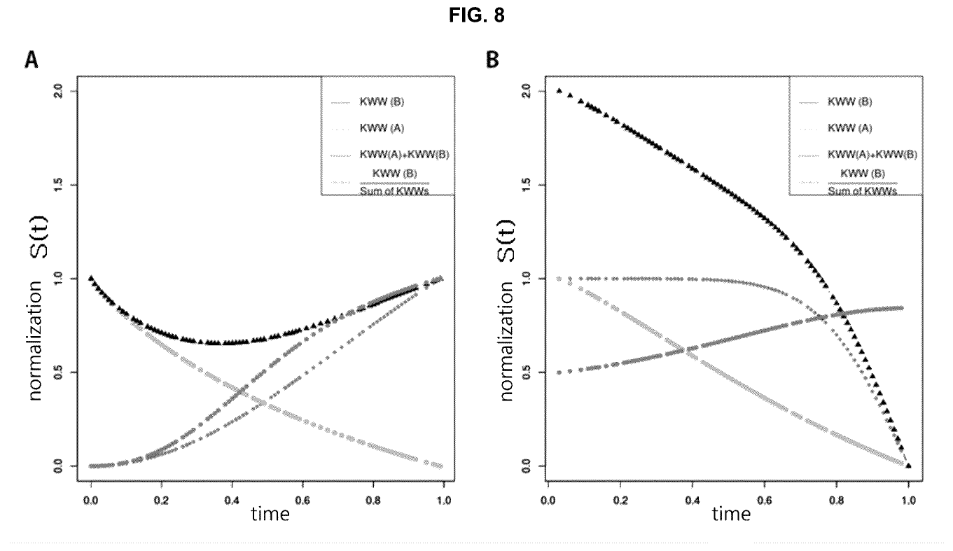

FIG. 7 illustrates two survival curves when a transformation equation is applied in step S430 illustrated in FIG. 4, and FIG. 8 illustrates relative ratios of the graphs illustrated in FIG. 7.

As illustrated in FIG. 7, it is assumed that exp(−t^{α}) is a survival curve of a disease factor, exp(−t^{β}) is a survival curve of an intrinsic factor, and influence of the two curves is 1:1. 67

Then, the two curves are min-max-normalized and respectively referred to as KWW (A) and KWW (B), and a relative ratio of KWW (B) is represented in FIG. 8 below.

Referring to FIG. 8, (α1×exp(−t^{α}) is normalized to acquire KWW (A), (β1×exp(−t^{β})+e) is normalized to acquire KWW (B), and then the acquire KWW (A) and KWW (B) are inserted into

$$\frac{KWW ( B )}{KWW ( A ) + KWW ( B )}$$

to acquire a graph against time.

FIG. 8 (A) is a graph obtained by using the graph illustrated in FIG. 7 (A), and FIG. 8 (B) is a graph obtained by using the graph illustrated in FIG. 7 (B).

In addition, the relative ratios of KWW (B) illustrated in FIG. 8 (A) and (B) are calculated by using the sigmoid equation described in Equation 3 below.

$$y = d + \frac{a - d}{{( 1 + exp ( b ( x - c ) ) )}^{g}} \qquad [ Equation 3 ]$$

Here, a is a maximum value, b is a slope factor, c is a position parameter, d is a minimum value, and g represents an asymmetry factor.

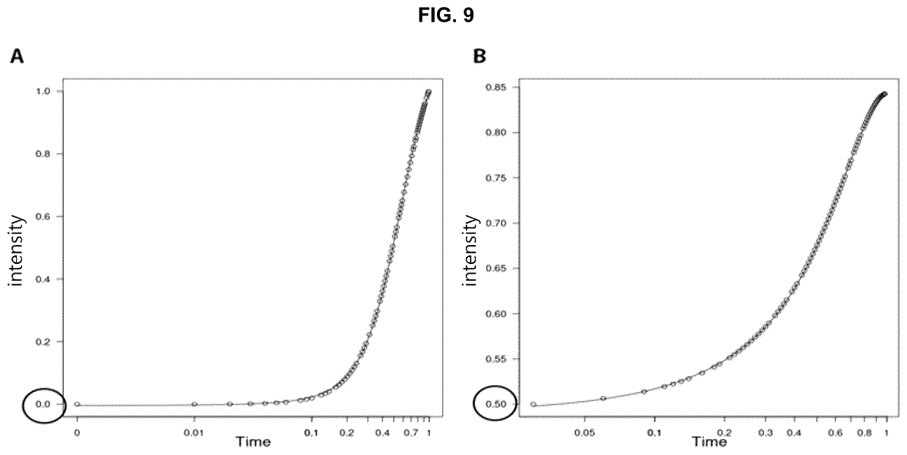

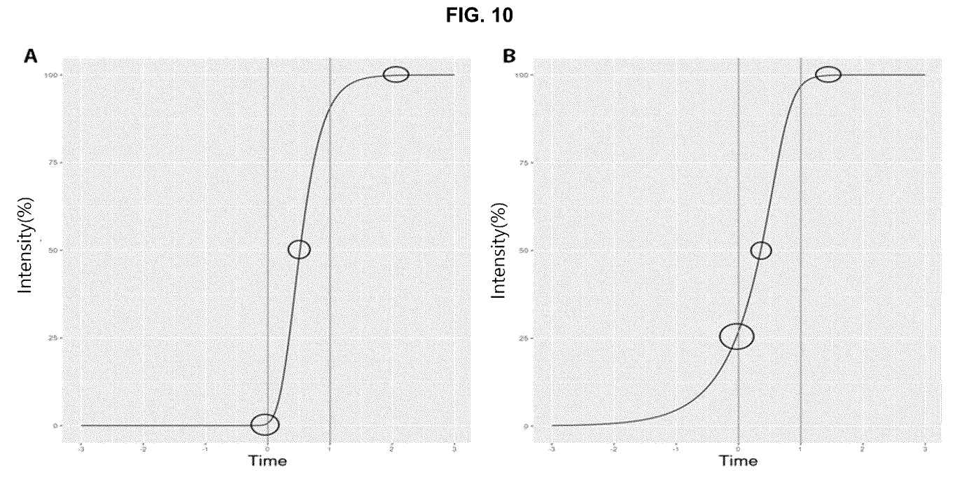

FIG. 9 illustrates graphs of intensity according to a relative ratio of KWW (B) obtained from FIG. 8, and FIG. 10 illustrates examples of a method of acquiring intensity for a period of −3 to 3 from a sigmoid curve derived by using the data collected in a period of 0 to 1.

As illustrated in FIG. 9, the analysis unit 330 converts a relative ratio to a percentage by applying a sigmoid equation to the relative ratio. At this time, the converted percentage is defined as “intensity”.

As illustrated in FIG. 10 (A), referring to a sigmoid curve, when the relative ratio of exp(−t^{β}) is 0, the intensity at 0 time corresponds to approximately 0%.

In addition, as illustrated in FIG. 10 (B), when the relative ratio of exp(−t^{β}) is 0.5, the intensity at 0 time corresponds to approximately 25%.

When step S430 is completed, the analysis unit 330 classifies restricted mean time lost (RMLT) according to a change in time (S440).

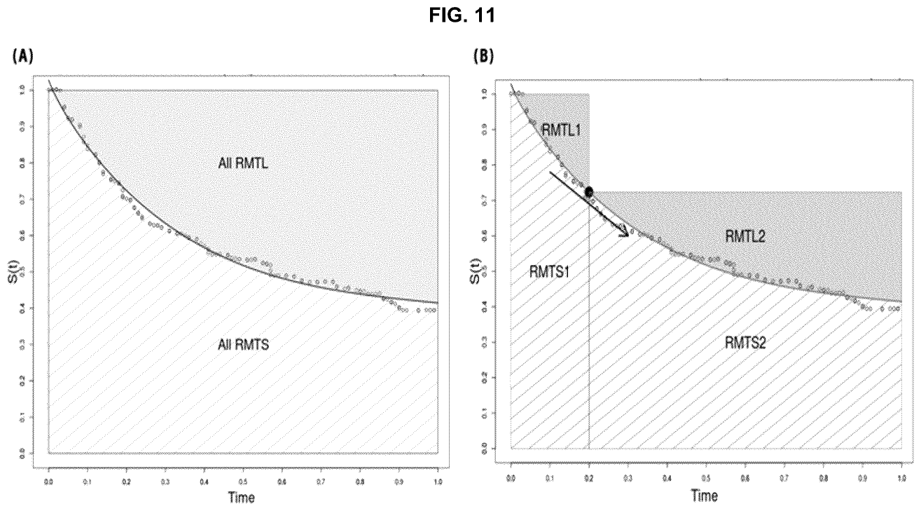

FIG. 11 illustrates graphs of the restricted mean time lost classified in step S430 illustrated in FIG. 4.

As illustrated in FIG. 11, the analysis unit 330 sets a specific point in time of an expected value of occurrence for death or relapse, and classifies the restricted mean time lost into a first restricted mean time lost (RMLT1) and a second restricted mean time lost (RMLT2) based on the set specific point in time.

At this time, the expected value of occurrence of an event corresponding to the entire observed time, that is, the time from 0 to 1, is defined as all restricted mean time lost (RMLT).

Then, the analysis unit 330 selects a certain section of the survival curve based on time, intensity, and restricted mean time lost (RMLT) (S450).

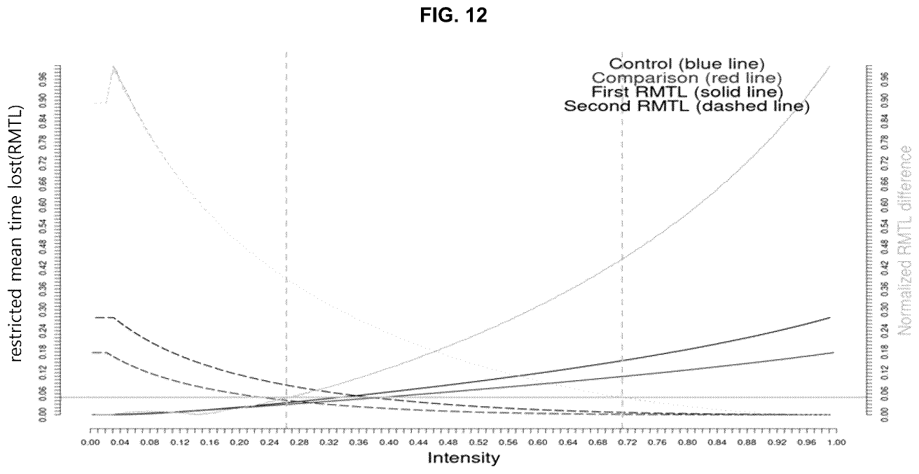

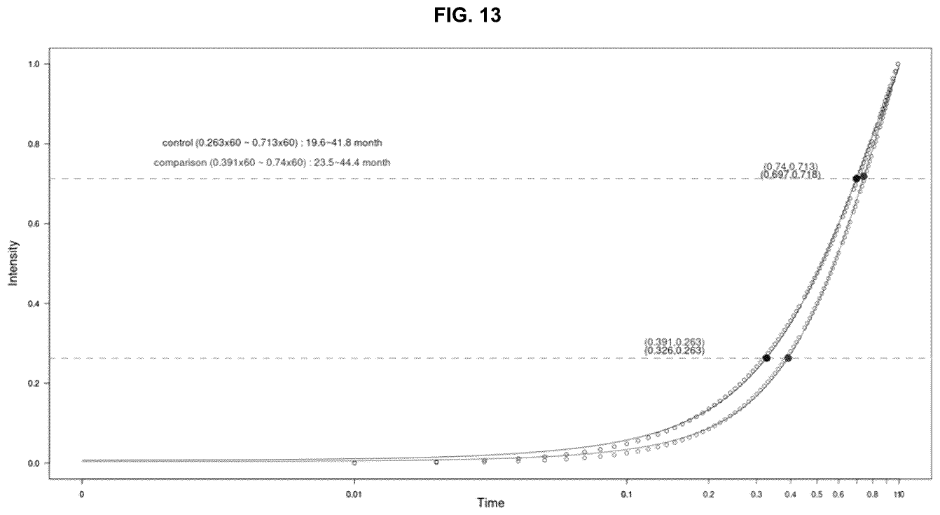

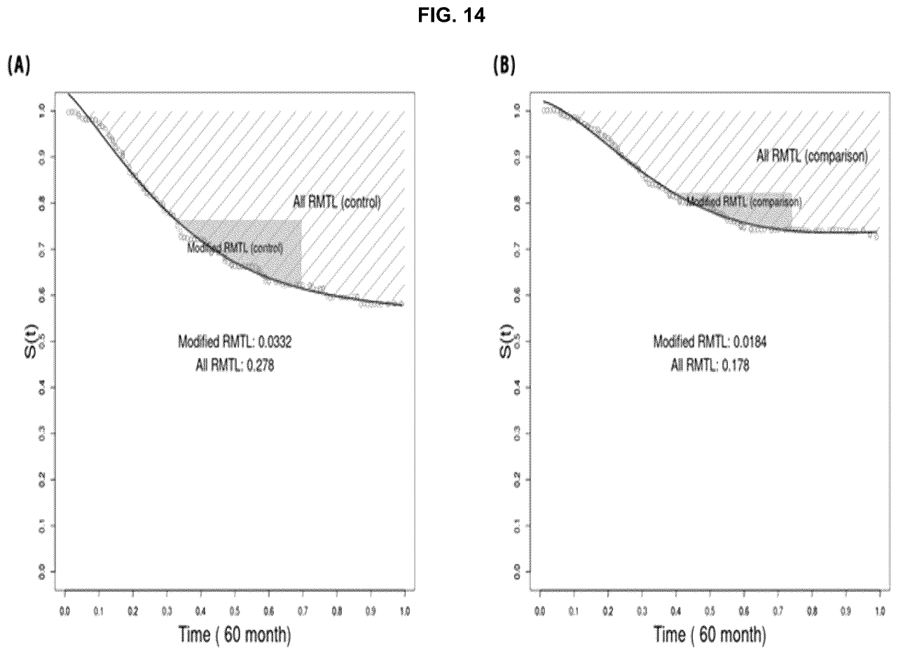

FIG. 12 is a graph showing a difference between intensity of a contrast group and a comparison group, the first restricted mean time lost (RMLT1), the second restricted mean time lost (RMLT2), and the restricted mean time lost (RMLT) in a clinical trial for comparing survival rates of chemoradiation and radiation therapy, FIG. 13 is a graph showing the time according to intensity in the restricted mean time lost illustrated in FIG. 12, and FIG. 14 illustrates graphs obtained by calculating all RMTL and modified RMTL of a contrast group and a comparison group illustrated in FIG. 13.

As illustrated in FIG. 12, the analysis unit 330 analyzes intensity and the restricted mean time lost (RMLT) to select a section where there is no difference in the restricted mean time lost (RMLT) for the intensities of the comparison group and the contrast group of a survival curve.

Then, the analysis unit 330 selects a section where the intensity is less than 0.4 from the first restricted mean time lost (RMLT1) and a difference in the first restricted mean time lost (RMLT1) of the comparison group and the contrast group is less than 5% of a maximum value.

In addition, the analysis unit 330 selects a section where the intensity is less than 0.4 from the second restricted mean time lost (RMLT2) and a difference in the second restricted mean time lost (RMLT2) of the comparison group and the contrast group is less than 5% of a maximum value.

Then, as illustrated in FIG. 13, the analysis unit 330 obtains the time corresponding to the intensity of each point in time by using a sigmoid curve and calculates the entire restricted mean time lost (RMLT) and the modified restricted mean time lost (RMLT) for the contrast group and the comparison group based on the obtained time.

In addition, the analysis unit 330 calculates a ratio

$$( \frac{comparison}{control} )$$

for the restricted mean time lost (RMLT). As illustrated in FIG. 14, a ratio of the total restricted mean time lost is 0.64, and a ratio of the modified restricted mean time lost is 0.553.

In this way, the survival curve generating system according to the present invention may theoretically suggest the existence of other groups in the existing survival curve, and may specify groups that are not related to the applied effect in the survival curve of a randomized clinical trial for evaluating a treatment effect of cancer, and thereby accurately and efficiently evaluating an effect of the randomized clinical trial for evaluating the treatment effect of cancer.

In addition, the survival curve generating system according to the present invention may provide meaningful information on the comparison between a comparison group and a contrast group by separating a treatment refractory group in results of survival curves, and may re-evaluate the groups that do not show statistically significant differences during an observation period.

The present invention is described with reference to the embodiments illustrated in the drawings, but the embodiments are merely examples, and those skilled in the art will understand that various modifications and equivalent other embodiments may be derived therefrom. Therefore, the true technical protection scope of the present invention should be determined by the technical idea of the following patent claims.

### REFERENCE SIGNS LIST

300: survival curve generating system

310: data collection unit

320: survival curve construction unit

330: analysis unit

1. A survival curve generating system using an exponential function, the survival curve generating system comprising:
a data collection unit that collects data obtained from a clinical trial for cancer;
a survival curve construction unit that generates a survival curve by using a survival curve model including multiple exponential functions based on the collected data; and
an analysis unit that calculates a relative ratio of one exponential function included in the survival curve model, obtains intensity from a sigmoid curve derived using the relative ratio, and separates a patient group based on the intensity.

2. A survival curve generating method using a survival curve generating system, the survival curve generating method comprising:
a step of collecting data obtained from a clinical trial for cancer;
a step of modeling a survival curve by using the collected data;
a step of calculating intensity by applying a sigmoid equation to a relative ratio of the survival curve;
a step of classifying restricted mean time lost (RMLT) according to a time change; and
a step of selecting a section of the survival curve by using time, intensity, and the restricted mean time lost (RMLT).

3. The survival curve generating method of claim 2, wherein, in the step of modeling the survival curve, the survival curve is modelled by using a KWW function generated by using an equation below,
$$S ( t ) = α 1 \times exp ( - {t}^{α} ) + β 1 \times exp ( - {t}^{β} ) + e ( β \geq 1 ⋂ α > 0 ⋂ β - α > 0 , c = β - α )$$

4. The survival curve generating method of claim 3, wherein, in the step of modeling the survival curve, a range of α is set from 0.01 to 10 at 0.01 intervals and a range of β is set from 1 to 10 at 0.1 intervals.

5. The survival curve generating method of claim 2, wherein, in the step of calculating the intensity, KWW (A) is obtained by normalizing α1×exp(−t^{α}), KWW(B) is obtained by normalizing β1×exp(−t^{β})+e and then a graph for time is obtained by inserting the obtained KWW (A) and KWW (B) into
$$\frac{KWW ( B )}{KWW ( A ) + KWW ( B )} .$$

6. The survival curve generating method of claim 2, wherein, in the step of calculating the intensity, the intensity is calculated by applying a sigmoid equation of an equation below to a relative ratio for KWW (B),
$$y = d + \frac{a - d}{{( 1 + exp ( b ( x - c ) ) )}^{g}}$$
where a is a maximum value, b is a slope factor, c is a position parameter, d is a minimum value, and g represents an asymmetry factor.

7. The survival curve generating method of claim 2, wherein, in the step of classifying the restricted mean time lost (RMLT), a specific point in time for an expected value of occurrence for death or relapse is set, and the restricted mean time lost (RMLT) is classified into first restricted mean time lost (RMLT1) and second restricted mean time lost (RMLT2) based on the set specific point in time.

8. The survival curve generating method of claim 2, wherein, in the step of selecting the section of the survival curve, the intensity and the restricted mean time lost (RMLT) are analyzed to select a section in which there is no difference in the restricted mean time lost (RMLT) for intensities of a comparison group and a contrast group of the survival curve.
